(19) United States
(12) Patent Application Publication
(10) Pub. No.: US 2008/0027128 A1
(43) Pub. Date: Jan. 31, 2008

(54) Duloxetine HCL polymorphs

(76) Inventors: Santiago Ini, Haifa (IL); Tamas Koltai, Netanya (IL); Shalom Shabat, Yavne (IL)

Correspondence Address: KENYON & KENYON LLP, ONE BROADWAY, NEW YORK, NY, 10004, US

(21) Appl. No.: 11/805,625

(22) Filed: May 23, 2007

Related U.S. Application Data
(60) Provisional application No. 60/808,094, filed on May 23, 2006.

(51) Int. Cl. A61K 31/381; A61P 25/04; A61P 25/24; C07D 333/08

(52) U.S. Cl. 514/438; 549/75

(57) ABSTRACT

A crystalline form of duloxetine hydrochloride, pharmaceutical compositions of the crystalline form of duloxetine hydrochloride, and methods of preparing the crystalline form of duloxetine hydrochloride are provided.

### CROSS-REFERENCE TO RELATED APPLICATIONS

The present application claims the benefit of the following U.S. Provisional Patent Application No. 60/808,094 filed May 23, 2006, the contents of which is incorporated herein by reference.

### FIELD OF INVENTION

The present invention is directed to solid states of duloxetine HCl and methods of preparation thereof.

### BACKGROUND OF THE INVENTION

Duloxetine is a dual reuptake inhibitor of the neurotransmitters serotonin and norepinephrine, and has been found to have application for the treatment of stress urinary incontinence (SUI), depression, and pain management.

Polymorphism, the occurrence of different crystal forms, is a property of some molecules and molecular complexes. A single molecule, like duloxetine HCl, may give rise to a variety of crystalline forms having distinct crystal structures and physical properties like melting point, x-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different crystalline forms or polymorphs of the same pharmaceutical compounds can and reportedly do have different aqueous solubilities.

A method for the synthesis of duloxetine HCl is disclosed in U.S. Pat. No. 5,362,886. Duloxetine HCl is commercially available as CYMBALTA®.

US2006/0270859, a commonly assigned application, repeats the process of U.S. Pat. No. 5,362,886 and reports that the product is an anhydrous polymorphic form of duloxetine HCl, designated Form A. This form is reported to be characterized by X-ray powder diffraction peaks at about 9.6°, 13.9°, 18.1°, 18.9°, 20.9° and 23.4° 2θ±0.2° 2θ. The XRD pattern of Form A is illustrated in FIGS. 1 (wet) and 2 (dry) of the present application.

US2006/0270859 further discloses two forms of duloxetine HCl, including a crystalline form designated as Form B and amorphous form. The crystal Form is reported to be obtained by evaporating methanol from a solution having duloxetine HCl. The crystalline form is said to be characterized by peaks at about 11.1, 12.1, 14.9, 21.6 and 24.2±0.2° 2θ.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for polymorphic forms of duloxetine HCl.

### SUMMARY OF THE INVENTION

In one embodiment, the present invention provides duloxetine HCl solvate. Preferably, the duloxetine HCl solvate is an acetone solvate.

In another embodiment, the present invention provides crystalline duloxetine HCl, characterized by X-ray powder diffraction peaks at about 10.5°, 16.7°, 23.9°, 24.8°, and 27.7° 2θ±0.2° 2θ.

In another embodiment, the present invention provides a method of preparing the above duloxetine HCl crystal Form, comprising combining duloxetine, acetone and HCl to obtain duloxetine HCl crystal Form characterized by X-ray powder diffraction peaks at about 10.5°, 16.7°, 23.9°, 24.8°, and 27.7° 2θ±0.2° 2θ. Alternatively, the starting material may be a salt of duloxetine.

In another embodiment, the present invention provides a process of preparing duloxetine HCl crystal Form characterized by X-ray powder diffraction peaks at about 9.6°, 13.9°, 18.1°, 18.9°, 20.9° and 23.4° 2θ±0.2° 2θ, comprising drying duloxetine HCl crystal Form, characterized by X-ray powder diffraction peaks at about 10.5°, 16.7°, 23.9°, 24.8°, and 27.7° 2θ±0.2° 2θ.

In another embodiment, the present invention provides pharmaceutical compositions comprising duloxetine HCl crystal Form characterized by X-ray powder diffraction peaks at about 10.5°, 16.7°, 23.9°, 24.8°, and 27.7° 2θ±0.2° 2θ.

### BRIEF DESCRIPTION OF THE FIGURES

FIG. 1 illustrates the powder X-ray diffraction pattern for wet Duloxetine HCl (obtained by the procedure disclosed in U.S. Pat. No. 5,362,886).

FIG. 2 illustrates the powder X-ray diffraction pattern for dry Duloxetine HCl (obtained by the procedure disclosed in U.S. Pat. No. 5,362,886.

FIG. 3 illustrates the powder X-ray diffraction pattern for Duloxetine HCl Form C.

FIG. 4 illustrates the powder X-ray diffraction pattern for Duloxetine HCl Form A, obtained by drying Form C.

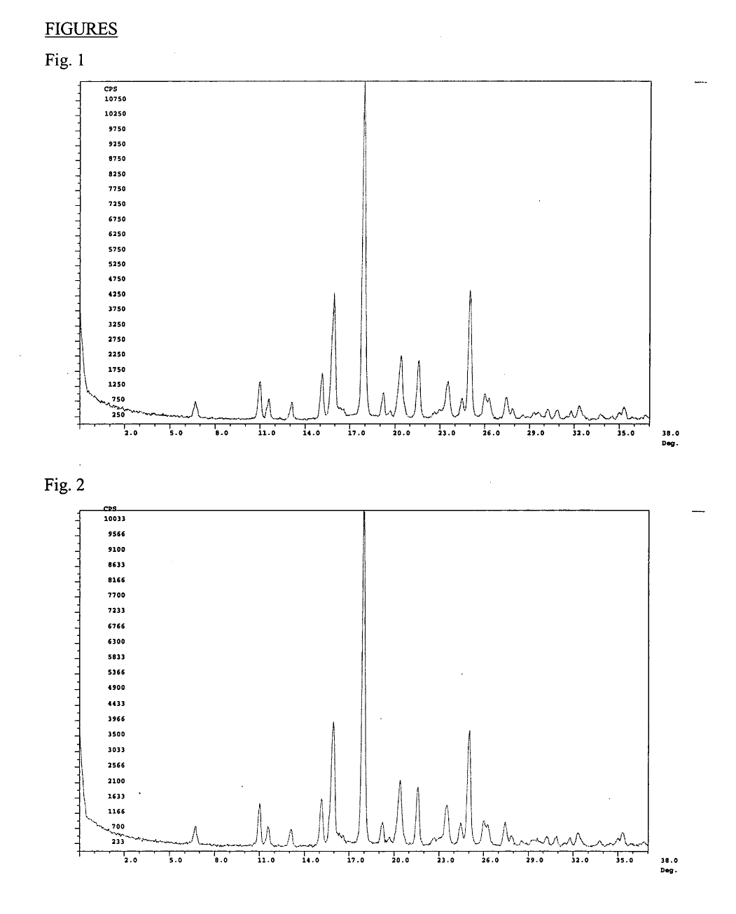

### DETAILED DESCRIPTION OF THE INVENTION

As used herein the term “anhydrous” refers to duloxetine HCl containing not more than 0.5% water/solvent by weight.

As used herein, “solvate” is meant to include any crystalline form which incorporates solvent in a level of more than about 1% by weight.

In one embodiment, the present invention provides duloxetine HCl solvate. Preferably, the duloxetine HCl of the present invention is acetone solvate.

In another embodiment, the present invention provides crystalline duloxetine HCl, herein defined as Form C, characterized by X-ray powder diffraction peaks at about 10.5°, 16.7°, 23.9°, 24.8°, and 27.7° 2θ±0.2° 2θ.

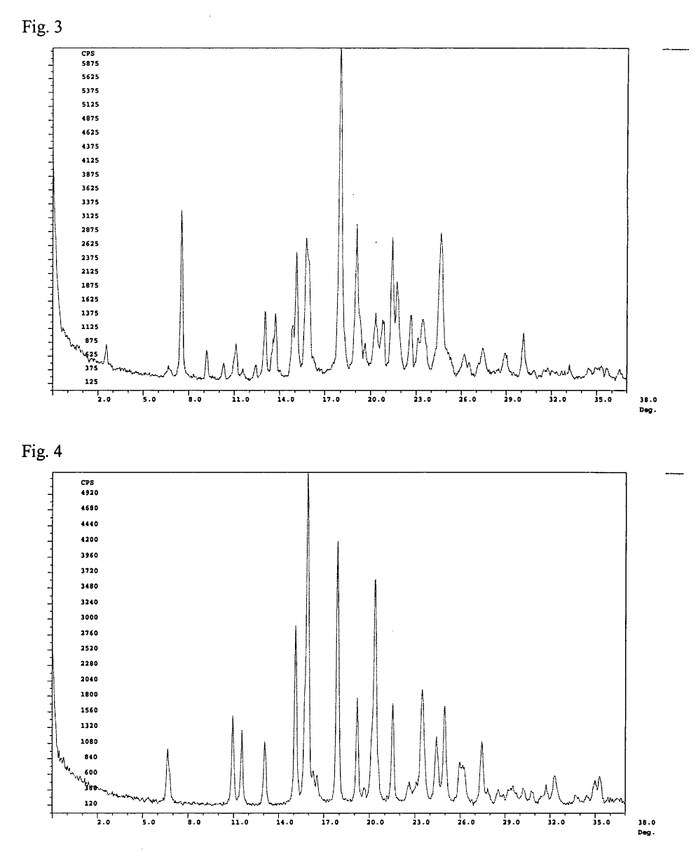

The crystalline form may be further characterized by an X-ray powder diffraction pattern with peaks at about 5.5°, 13.3°, and 15.4° 2θ±0.2° 2θ, substantially as depicted in FIG. 3.

Form C has a weight loss, as measured by TGA, of about 9% by weight. This corresponds to acetone solvate of duloxetine HCl.

The present invention further provides a method of preparing duloxetine HCl crystal Form C, comprising combining duloxetine, acetone and HCl to obtain duloxetine HCl.

The duloxetine starting material can be introduced into the reaction either as a base or in its HCl salt form. When the starting material is duloxetine HCl, the addition of HCl is not necessary.

In one embodiment, a solution of duloxetine in acetone is combined with HCl to obtain a solid, followed by the recovering duloxetine HCl crystal Form C.

Preferably, the solution is maintained while stirring at about room temperature.

Optionally, before combining the solution with HCl, the solution is seeded with duloxetine HCl. Preferably, the duloxetine HCl seeds are either Form A or Form C, most preferably, Form A.

Preferably, after seeding, the solution is further maintained for about 5 minutes to about one hour.

Preferably, the HCl combined is gaseous.

Preferably, HCl is bubbled into the solution, until a pH of about 3 to about 5 is obtained.

Preferably, after combining the solution with HCl the mixture is further maintained for about 5 minutes to about 30 minutes.

Duloxetine HCl crystal Form C may be recovered by any method known in the art, such as filtering. The recovered crystals can be further washed.

In another embodiment, the present invention provides a process of preparing duloxetine HCl crystal Form A, comprising drying Form C.

As one skilled in the art will appreciate, the time required to obtain duloxetine HCl crystal Form A will vary depending upon, among other factors, the amount of wet duloxetine HCl Form C to be dried and the drying temperature, and can be determined by taking periodic XRD's. Preferably, Form C is dried at about room temperature to about 70° C., at a pressure below about 1 atmosphere, more preferably below about 100 mm Hg, The time for drying is preferably at least 5 minutes, more preferably, for about 6 hours.

In another embodiment, the present invention provides pharmaceutical compositions comprising duloxetine HCl crystal Form C.

Pharmaceutical compositions may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups, and suspensions. Suitable forms of parenteral administration include an aqueous or non-aqueous solution or emulsion, while for rectal administration, suitable forms for administration include suppositories with hydrophilic or hydrophobic vehicle. For topical administration, the invention provides suitable transdermal delivery systems known in the art, and, for nasal delivery, there are provided suitable aerosol delivery systems known in the art.

In addition to the active ingredient(s), the pharmaceutical compositions of the present invention may contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. AVICEL®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. EUDRAGIT®), potassium chloride, powdered cellulose, sodium chloride, sorbitol, and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB®), and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease the release of the product from the die. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be died using any pharmaceutically acceptable colorant to improve their appearance and/or facilitate patient identification of the product and unit dosage level.

In liquid pharmaceutical compositions of the present invention, the active ingredient and any other solid excipients are suspended in a liquid carrier, such as water, vegetable oil, alcohol, polyethylene glycol, propylene glycol, or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar may be added to improve the taste.

Preservatives and chelating agents such as alcohol, sodium benzoate, butylated hydroxy toluene, butylated hydroxyanisole, and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate, or sodium acetate.

Selection of excipients and the amounts used may be readily determined by the formulation scientist based upon experience and consideration of standard procedures and reference works in the field.

The solid compositions of the present invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present invention is oral. The dosages may be conveniently presented in unit dosage form and prepared by any of the methods well known in the pharmaceutical arts.

Dosage forms include solid dosage forms like tablets, powders, capsules, suppositories, sachets, troches, and losenges, as well as liquid syrups, suspensions, and elixirs.

The dosage form of the present invention may be a capsule containing the composition, preferably a powdered or granulated solid composition of the invention, within either a hard or soft shell. The shell may be made from gelatin, and, optionally, contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended, and then further mixed in the presence of a liquid, typically water, that causes the powders to clump into granules. The granulate is screened and/or milled, dried, and then screened and/or milled to the desired particle size. The granulate may then be tableted or other excipients may be added prior to tableting, such as a glidant and/or a lubricant.

A tableting composition may be prepared conventionally by dry blending. For example, the blended composition of the actives and excipients may be compacted into a slug or a sheet, and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

### EXAMPLES

While the present invention is described with respect to particular examples and preferred embodiments, it is understood that the present invention is not limited to these examples and embodiments. The present invention, therefore, includes variations from the particular examples and preferred embodiments described herein, as will be apparent to one of skill in the art.

The X-ray diffraction diffractometer used to analyze and identify the crystalline forms of duloxetine HCl was a Scintag X-ray powder diffractometer model X'TRA, Cu-tube solid state detector. The sample holder was a standard round aluminum sample holder with a rough zero background quartz plate, having a cavity diameter of 25 mm and a depth of 0.5 mm. The scanning parameters were:
range: 2° to 40° 2θ;
scan mode: continuous scan;
step size: 0.05°; and
rate: 5°/minute.

To determine the weight loss by thermal gravimetric analysis (TGA), the sample was heated from about 25° C. to about 200° C. at a heating rate of about 10° C. per minute, while purging with nitrogen gas at a flow rate of 40 ml/min.

### Example 1

### Preparation of Duloxetine HCl Form C

A 100 L glass reactor equipped with mechanical stirrer, thermometer, and condenser was charged with 49.7 Kg of a solution of Duloxetine in toluene (87%). The solution was distillated under vacuum of 20-30 mm Hg till dryness. After cooling to room temperature, 63.65 liters of acetone were added, the solution was stirred at 25° C. for 45 minutes, and 10 g of Duloxetine hydrochloride were added. After 1 hour stirring, HCl was bubbled into the solution until the mixture reach pH 3, and the solution was stirred at the same temperature for half hour. The resulting solid was filtrated out, washed with acetone (9.5 liters), analyzed by XRD, and Form C was identified in the sample.

### Example 2

### Preparation of Duloxetine HCl Form C

A 100 L glass reactor equipped with mechanical stirrer, thermometer, and condenser was charged with a solution of Duloxetine in acetone (11.5%). The solution was stirred at 30° C. during 15 minutes and HCl was bubbled into the solution until the mixture reach pH 3 and cooled to 0° C. The resulting solid was filtrated out, washed with acetone (30 ml×3), and analyzed by XRD.

### Example 3

### Preparation of Duloxetine HCl Form A

Duloxetine HCl Form C was dried in a vacuum oven (27-34 mm Hg) at 47°-54° C. for 6 hours. The dry sample was analyzed by XRD, and Form A was identified in the sample (FIG. 4).

What is claimed is:

1. Crystalline Duloxetine HCl solvate.

2. The crystalline duloxetine HCl of claim 1, wherein the solvate is acetone solvate.

3. A crystalline form of duloxetine HCl characterized by an X-ray powder diffraction pattern with peaks at about 10.5°, 16.7°, 23.9°, 24.8°, and 27.7° 2θ±0.2° 2θ.

4. The crystalline form of claim 3, further characterized by an X-ray powder diffraction pattern with peaks at about 5.5°, 13.3°, and 15.4° 2θ±0.2° 2θ.

5. The crystalline form of claim 3, characterized by an X-ray powder diffraction pattern as depicted in FIG. 3.

6. The crystalline form of claim 3, having a weight loss, as measured by TGA, of about 9% by weight.

7. The crystalline form of claim 3, which is an acetone solvate.

8. A process for preparing duloxetine HCl crystal of claim 3 comprising combining duloxetine or a duloxetine salt, acetone and HCl to obtain duloxetine HCl crystal.

9. The process of claim 8, wherein the duloxetine salt is duloxetine HCl.

10. The process of claim 8, wherein a solution of duloxetine in acetone is combined with HCl to precipitate duloxetine HCl.

11. The process of claim 8, further comprising seeding before or after addition of HCl.

12. The process of claim 11, wherein seeding is carried out before addition of HCl.

13. The process of claim 8, wherein the HCl is gaseous.

14. The process of claim 8, wherein a pH of about 3 to about 5 is obtained after addition of HCl.

15. The process of claim 8, wherein recovery is carried out by filtration.

16. A process for preparing a crystalline form of duloxetine HCl characterized by an X-ray powder diffraction with peaks at about 9.6°, 13.9°, 18.1°, 18.9°, 20.9° and 23.4° 2θ±0.2° 2θ comprising drying the crystalline form of duloxetine HCl of claim 3.

17. The crystalline form of claim 16, wherein drying is carried out at about room temperature to about 70° C. and a pressure below about 1 atmosphere.

18. The process of claim 17, wherein drying is carried out at a pressure below about 100 mm Hg.

19. A pharmaceutical compositions comprising the crystalline form of duloxetine HCl of claim 3 and at least one pharmaceutically acceptable excipient.

20. A method of inhibiting reuptake of serotonin and norepinephrine in a mammal comprising administering the pharmaceutical composition of claim 20 to the mammal.

21. A process for preparing duloxetine HCl crystal of claim 3 comprising combining duloxetine HCl and acetone.
